(19) United States
(12) Patent Application Publication
(10) Pub. No.: US 2022/0000597 A1
(43) Pub. Date: Jan. 6, 2022

(54) EXTRA-ORAL AEROSOL CAPTURE HOOD

(71) Applicants: Bert Milford SIMPSON, JR., Greenville, SC (US); Theodore Kenneth HOFFMAN, Greenville, SC (US)

(72) Inventors: Bert Milford SIMPSON, JR., Greenville, SC (US); Theodore Kenneth HOFFMAN, Greenville, SC (US)

(21) Appl. No.: 17/365,127

(22) Filed: Jul. 1, 2021

Related U.S. Application Data
(60) Provisional application No. 63/046,931, filed on Jul. 1, 2020.

(51) Int. Cl. A61C 19/00; A61C 1/08

(52) U.S. Cl. CPC A61C 19/007; A61C 1/088

(57) ABSTRACT

An extra-oral capture hood includes a first cone that provides a first path for a vacuum product through the extra-oral capture hood. A second cone concentric with the first cone and disposed around the first cone provides a second path for the vacuum product through the extra-oral capture hood, the second path being located between the first cone and second cone. A disconnect is connected to at least one of the first cone or second cone, the disconnect being configured to connect the extra-oral capture hood to a duct, a valve or other component of a capture system.

### BACKGROUND

With the onset of COVID-19, trips to the dentist have become a cause for concern in light of the fact that the patient is receiving treatment with his or her mouth open for a prolonged period of time. In the event of a restorative, hygienic, or surgical procedure, pressurized water may be sprayed into the patient's mouth, a powered scaling device, dental drill, compressed air device, etc. may be used, and other procedures (cavity fillings, crowns, restorative procedures, surgical procedures, preventative procedures, etc.) may be performed that result in water vapor, sprays, exhaled breath, volatile organic compounds, dust, restoration particulate, tooth structure, adhesives, etc. (collectively, “dental aerosols”) being released/expelled from the patient's mouth and/or nose as a result of the dental procedure. These dental aerosols may include viruses, bacteria, and other biohazards that could infect or otherwise injure others in and around the oral care provider's office (i.e. dentists, dental hygienists, orthodontists, periodontists, endodontists, oral surgeons, prosthodontists, other patients, employees, etc.). In fact, the risk is not limited to the room in which procedures are performed. Dental aerosols are able to circulate within the entire provider's office (and potentially other businesses close by), which could infect, for example, patients waiting in a waiting room, receptionists, etc. As a result of this risk for infection, the CDC has recently recognized four dental occupations as falling within the top ten highest risk occupations.

Presently, there are only a few limited ways to capture dental aerosols, and these have their drawbacks. High vacuum, low volume intraoral suction devices that include a tube that is inserted into a patient's mouth are intended to remove saliva, water, etc. from the mouth have been in use in dental offices for generations. These suction devices do not significantly limit the dental aerosols that are produced by the patient. Extra-oral devices using high vacuum, low volume suction systems have been recently introduced into the dental equipment market, but these systems do not have adequate flow capacity to effectively capture the quantity of extra-oral aerosols produced during a procedure. Stand-alone filtration units that are placed at or near a patient's location (i.e. point-of-use devices) have been developed, but these devices are awkward to use, take up a large amount of space in the room, are noisy, and discharge air back into the room. Further, these point-of-use devices do not typically separate liquid from non-liquid dental aerosols, which can result in bacteria, mold, etc. being present in the unit. If the filtration units are not functioning properly or not cleaned as required, the filtration units could discharge contaminated air back into the room. Further, these filtration units usually serve only a single patient, or perhaps two patients.

What is needed is a centralized extra-oral dental aerosol capturing system that is easy to use, that effectively captures dental aerosols such that it limits or prevents the spread of contaminants, and that discharges into a safe location, preferably outside of the rooms in which dental procedures are conducted. A more energy efficient, dental aerosol capture system utilizing a high efficiency capture hood may reduce the number of vacuum units required to capture dental aerosols from two or more procedure rooms, may reduce the power requirements needed to capture aerosols from a single capture hood and/or may reduce the need for significant modifications to the dental office electrical distribution system.

### SUMMARY

In one embodiment, described herein, an extra-oral capture hood includes a first cone that provides a first path for a vacuum product through the extra-oral capture hood. A second cone concentric with the first cone and disposed around the first cone provides a second path for the vacuum product through the extra-oral capture hood. The second path is located between the first cone and second cone. A disconnect is connected to at least one of the first cone or second cone, the disconnect configured to connect the extra-oral capture hood to a duct, a valve or other component of a capture system. The first cone may include a first end opposite the disconnect, and the second cone may include a second end opposite the disconnect, the second end extending past the first end. The second cone may be flared between the first end and the second end. Vacuum channels may be present between the first cone and second cone, the vacuum channels further defining the second path. The vacuum channels may have a curved path. A material used to form the vacuum channels may connect the first cone to the second cone. The extra-oral capture hood may further include a light. The light may include a ring of LED lights located around the second cone. The extra-oral capture hood may further include a handle. The disconnect may include a quick disconnect.

According to another embodiment, described herein, an extra-oral capture system for capturing a vacuum product includes a hood comprising a first cone, a second cone and a disconnect. The first cone defines a first path for the vacuum product, and a second path for the vacuum product is defined between the first cone and second cone. A valve is connected to the disconnect, the valve configured to adjust the flow of the vacuum product. The extra-oral capture system further includes a duct connected to the valve and a vacuum configured to provide a vacuum pressure to the duct, the vacuum pressure causing the vacuum product to enter the hood. The extra-oral capture system may further include a second hood connected to the duct. The hood may be located in a first room, and the vacuum is located in a second room. The first cone may include a first end opposite the disconnect, and the second cone may include a second end opposite the disconnect, the second end extending past the first end. The second cone may be flared between the first end and the second end. The hood may include a light. Vacuum channels may be present between the first cone and the second cone, the vacuum channels further defining the second path. The vacuum channels may be curved. A material used to form the vacuum channels may connect the first cone to the second cone. The extra-oral capture system may further include a reservoir including a decontamination fluid, the reservoir being configured to store the hood in the decontamination fluid.

### BRIEF DESCRIPTION OF THE DRAWINGS

FIG. 1A illustrates a non-limiting example environment in which the systems and/or methods described herein may be implemented.

FIG. 1B and 1C illustrate a non-limiting example embodiment of the collection hood of FIG. 1A.

FIG. 2 illustrates an example embodiment of a capture system in which the systems and/or methods described herein may be implemented.

FIG. 3 illustrates an alternative environment in which the systems and/or methods described herein may be implemented.

### DETAILED DESCRIPTION

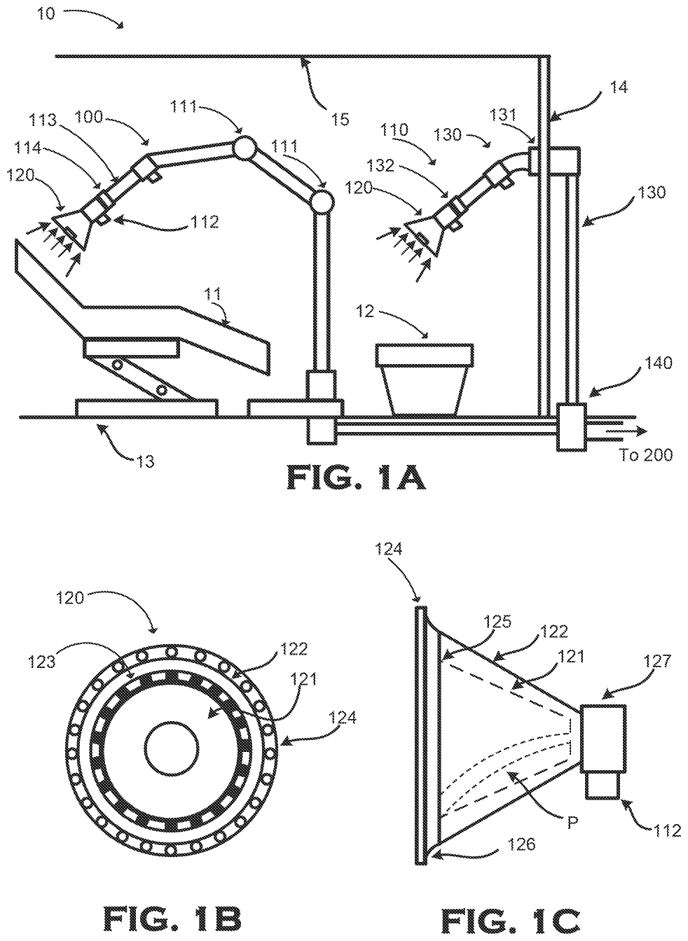

FIGS. 1A through 3 are attached hereto and incorporated herein by reference. The following detailed description refers to the accompanying FIGS. 1A through 3. The same reference numbers in different figures may identify the same or similar elements.

The systems, methods, technologies and/or techniques (hereinafter, the “systems and methods”), described herein, may include an extra-oral dental aerosol capture system that captures dental aerosols (i.e. exhaled breath, spray from aerosol generating armamentarium, cleaning, adjustments, enamels, adhesives, filling materials, VOCs, etc.) that are created by a patient during a dental visit or treatment by another provider (e.g. orthodontists, periodontists, endodontists, oral surgeons, prosthodontists, etc.) and are captured outside of the mouth of the user. As an example, the systems and methods described herein are explained in reference to dental procedures, but the systems and methods described herein are applicable to any provider associated with dental aerosols.

The dental aerosol capture system may include a capture hood that is positioned at or near the face of a dental patient. In one embodiment, the dental hood includes a light and is positioned approximately 6 inches in front of the patient's mouth and provides supplemental oral cavity illumination, which may supplement and/or replace a traditional dental light. The dental hood may be operably connected to a vacuum unit, which may cause a suction at the dental hood. As a result of the suction created at the dental hood, dental aerosols from the patient may be drawn into the hood and processed by the remaining components of the capture system.

The dental hood may generate an area, or volume, which is subject to the negative vacuum pressure, herein referred to as a vacuum field, in which a flow of air, dental aerosols, etc. are directed into the dental hood. The vacuum field may partially and/or completely envelope the head of the patient. Enveloping the head of the patient in this way may ensure that the dental aerosols from the patient are captured by the capture system. The vacuum field may generate a vacuum product (i.e. all of the air, dental aerosols, etc. that flow to the capture system), which may flow to the capture system and be treated as further described herein. The vacuum field may include a center portion which may generate a generally uniform velocity of air flowing into the capture system. The vacuum field may further include a perimeter portion which may generate a higher velocity of air flowing into the capture system than the inner portion. The perimeter portion may act as a barrier that limits and/or prevents dental aerosols from the patient from escaping the vacuum field, making the dental procedure safer for others.

The dental hood may be designed in a variety of ways and in a variety of shapes to optimize the vacuum field. The vacuum field may take on different properties for different embodiments, and the vacuum field anticipated by the present disclosure should not be limited to that described with regard to the example embodiments described herein. The dental hood may be optimized for specific conditions required by a dentist office. In one embodiment, the dental hood is formed from a pair of cone-shaped hood devices that create the inner portion and the perimeter portion of the vacuum field. Vacuum channels may be present between the pair of cone-shaped hood devices, which may be used to generate the perimeter portion of the vacuum field. The vacuum channels may correspond to channels or pathways through which the vacuum product, or a portion thereof, is suctioned to create the perimeter portion of the vacuum field. The vacuum channels may include curved pathways that create vortices and/or a swirling flow pattern in the perimeter portion, which may help form a barrier that limits and/or prevents the dental aerosols from escaping the vacuum field. The cones may be coaxial, which may cause the inner portion and/or the perimeter portion of the vacuum field to flow coaxial as well. The dental hood may be easily removed from the rest of the dental capture system for cleaning, changing, etc. Substitute dental hoods may be present in the dental office for easy change out, such as when a new patient arrives.

The capture system may include one or more dental hoods in a dental procedure room connected to other components of the capture system, which are ideally located partially and/or completely outside of the procedure room (i.e. outside, in a service closet, in a separated space, etc.). Additionally, or alternatively, the capture system may be located inside of the procedure room but may discharge outside of the procedure room. The capture system may include a separator, which may separate the liquid dental aerosol components from the non-liquid portions captured by the vacuum field. The separator may use any known type of water separator(s), such as a p-trap, in-line filters, scrubbers, bowl filters, etc. The separator may include a decontaminator that may treat the vacuum product to kill any viruses, bacteria, etc. and/or remove any contamination. In one embodiment, the decontaminator corresponds to a UV-C light unit, which may decontaminate the vacuum product using ultraviolet-c light, which is known to kill certain viruses, bacteria, fungus, etc. by irradiating them with UV-C light. Liquid portions of the vacuum product may be removed by the separator, which may drain the liquid portions of the vacuum product into the sewer, outside, etc.

The capture system may also, or alternatively, include a vacuum. The vacuum may generate the vacuum, or negative pressure, needed to create the vacuum field to capture the dental aerosols and/or to draw the vacuum product through the separator. The vacuum may be located downstream and/or upstream of the separator. The vacuum may correspond to a high capacity unit that may service a number of dental hoods. The vacuum may be configured to deliver a high capacity (i.e. 50 cubic feet per minute, 100 cfm, 250 cfm, etc.) low vacuum pressure (i.e. 1 inch mercury, 2 inches mercury, 4 inches mercury, etc.) at each dental hood connected to the capture system. This high capacity low vacuum pressure vacuum field may permit a dentist, dental hygienist, etc., to perform a dental procedure on the patient without being obstructed by the vacuum field and may also, or alternatively, limit and/or prevent dental aerosols from the patient from reaching the dentist, dental hygienist, etc. Further, the vacuum field may limit and/or prevent the exhaled breath and other contaminants from the dentist, dental hygienist, etc. from reaching the patient.

The capture system may further include a filter, which may remove particulates from the vacuum product. The filter may correspond to any type of known air filter (i.e. a HEPA filter, ionized filter, etc.) and may be placed at any location in the capture system. In one embodiment, the filter is located after the separator and before the vacuum in the capture system. In this embodiment, the vacuum product is decontaminated before it moves through the filter, which limits and/or prevents the presence of live contamination in the filter.

The capture system may also, or alternatively, include an exhaust unit. The exhaust unit may be located upstream and/or downstream of the vacuum and may include a second decontaminator. The second decontaminator may further decontaminate the vacuum product using, for instance, a second UV-C light emitter, etc. The exhaust unit may also, or alternatively, include a diffuser, which may diffuse the vacuum product so that it can be more effectively treated by the second decontaminator (i.e. may limit and/or prevent streams of high flow in favor of uniform flow, which may be more effectively treated). After the vacuum product exits the exhaust unit (or the vacuum, as designed), the vacuum product may be fully cleaned and ready to be exhausted into, for instance, a storage room, outside, etc.

The capture system may be centralized, meaning that the components other than the capture hood, ductwork and related equipment are located in a centralized area, generally away from the room or rooms in which the patients, or other persons, are receiving the treatment from one or more providers. Centralizing these components may allow multiple hoods to be powered from the centralized location, which may limit noise in the rooms in which procedures are taking place, may make it easier to maintain the capture system, may be cleaner, safer, etc.

A method of using the capture system may include placing the dental hood at or near the face of a dental patient. The method may further include operating the capture system to generate a vacuum field using the vacuum. The method may further include creating a vacuum product, via the vacuum field, to capture dental aerosols from the patient in a vacuum product. The method may further include passing the vacuum product through a separator to remove liquid from the vacuum product and/or to decontaminate the vacuum product. The method may further include filtering the vacuum product to remove particulates. The method may further include exhausting the vacuum product through an exhaust unit, which further decontaminates the vacuum product. The method may further include first diffusing the vacuum product before decontaminating the vacuum product in the exhaust unit. A vacuum may draw the vacuum product through the capture system.

FIGS. 1A illustrates a non-limiting example environments in which the systems and/or methods described herein may be implemented. Environment 10 may correspond to a dental procedure room that includes a dental chair 11, a reservoir 12, a floor 13, a wall 14, a ceiling 15, a first collector 100, and a second collector 110. The structures, collectors and components described in relation to FIGS. 1A are provided for explanatory purposes only, and the disclosure herein is not intended to be limited to the components and arrangement reflected in FIG. 1A. There may additional components, fewer components and/or differently arranged components than what is shown in FIG. 1A. Also, in other implementations, one or more of the components of FIG. 1A may perform the function of one or more other components of FIGS. 1A. Further, the capture system described herein is used in connection with a dentist office, but many other implementations are anticipated by the disclosure herein, including uses in, for instance, salons, doctor's offices, facial care centers, etc.

First collector 100 and second collector 110 provide a dental hood 120 to a patient (not shown). Dental hood 120, which will be further described with respect to FIGS. 1B and 1C, provides a vacuum field at or around the face of the user, which may capture the dental aerosols from the user. First collector 100 may further include pivots 111, handles 112, duct 113 and valve 114. Dental hood 120 may be connected to duct 113, which may carry the dental aerosols outside of the environment 10 (i.e. below floor 13, past wall 14, etc.) to the remaining portions of capture system 200. Additionally, or alternatively, dental hood 120 may be connected to valve 114, which may be located at the end of duct 113 (connected to duct with a permanent fitting, quick disconnect fitting, or other known ways of connection). Valve 114 may correspond to a shut off valve and/or flow control valve (ball valve, gate valve, saddle valve, etc.). Valve 114 may include a quick disconnect coupling disposed proximate the hood to allow hood 120 to be easily attached/removed (for cleaning, etc.). Additionally, or alternatively, duct 113 may include the quick disconnect coupling to quickly detach hood 120 and/or valve 114. Valve 114 may be closed when hood 120 is removed, which may limit and/prevent vacuum pressure from being lost. Additionally, or alternatively, valve may be closed when hood 120 is not being used, such as when other collector(s) are being used in an office and when the other collector(s) receive negative vacuum pressure from the same system. Additionally, or alternatively, valve 114 may be adjusted to increase and/or decrease the flow of the vacuum product (i.e. opened further to increase flow, closed further to limit flow). In this way, the flow of the vacuum product can be adjusted to suit the needs of a user and/or to ensure adequate flow when multiple hoods are being serviced by a centralized system. Pivots 111 may allow a user (dental hygienist, dentist, etc.) to turn/twist/etc. first collector 100 to place first collector 100 in an ideal location for performing a dental procedure on a patient. An ideal location for a dental procedure may correspond to dental hood 120 being far enough away from the patient to stay out of the way of the user/dental procedure provider while still providing a vacuum field that captures most and/or all of the dental aerosols generated by the patient. The user may adjust the position using handle 112. Handle 112 is depicted on hood 120, but may be present on other features of first collector 100, such as duct 113 or valve 114.

Duct 113 may extend from dental hood 120 or valve 114 to the remaining portions of the capture system (such as capture system 200, or a simpler system including at least a vacuum) to allow the vacuum product to be processed by the capture system as described herein. Duct 113 may correspond to ductwork, tubing, etc. that may withstand the vacuum created by the capture system and may provide the path through which the vacuum product reaches the remaining components of the capture system. As shown in FIG. 1A, dental hood 120 may be placed at or near the location where a dentist light, a typical fixture, is normally located. Additionally, a reservoir 12 may include decontamination fluid (i.e. a cold sterilization unit) and one or more additional dental hoods 120, which may be used for subsequent patients (i.e. the dental hoods are changed for new patients, after a number of uses, etc.).

Second collector 110 may be the same as, or similar to, first collector 100, except that, as shown, second collector 110 extends from the wall 14, rather than the floor 13. However, collectors may be positioned in a variety of places and configurations. For example, and not limitation, collectors may extend from a chair, such as chair 11 (e.g. the 2″ mounting hub of a typical dentist chair, etc.), the floor 13, the wall 14, the ceiling 15, etc. Collectors may be in a single room, multiple rooms, etc. Second collector 110 may include a dental hood 120, duct 130 and fitting 131. Duct 130 may include a valve 132, which may function the same as, or similar to, valve 114. Fitting 131 may provide a way to connect a collector to the capture system. Fitting 131 is shown on wall 14, but may be located on floor 13, the ceiling, etc. Several fittings, like fitting 131, may be plumbed into a dentist's office when installing a centralized capture system. A manifold 140 may connect duct 113 and duct 130 to a single duct that provides the vacuum product from first collector 100 and second collector 110 to the rest of capture system 200, which allows a central capture system to service multiple hoods located in multiple rooms.

FIGS. 1B and 1C depict the dental hood 120 of FIG. 1A. Dental hood 120 is merely an example embodiment of a hood that may be used to capture the aerosols described herein. Dental hood 120 may provide a vacuum field to a patient, such as when the patient is in a dental chair, to capture the aerosols emitted by the patient. As shown in FIGS. 1B and 1C, dental hood 120 may include a first cone 121 and a second cone 122. First cone 121 and second cone 122 are referred to as different components, but first cone 121 and second cone 122 may be formed from the same piece of material (i.e. a single molded or welded part, etc.), which may include some and/or all of the other components of hood 120. First cone 121 may correspond to an open-style hood portion through which the vacuum product, or a portion thereof, may pass when a vacuum is applied to dental hood 120 as described herein. Additionally, or alternatively, one or more diffusers, bafflers or other flow controllers (not shown) may be placed within first cone 121 to limit and/or regulate the vacuum flow through first cone 121. First cone 121 may provide the inner portion of the vacuum field as described herein. First cone 121 may define a first path inside of first cone 121, which may define an inner portion of a vacuum field. The inner portion of vacuum field may be directed to capturing aerosols from a patient. Second cone 122 may be located around first cone 121 and may be coaxial with first cone 121. Second cone 122 may provide a second path, between first cone 121 and second cone 122, which defines the outer portion, or boundary layer, of the vacuum field. Vacuum channels 123 may be located between first cone 121 and second cone 122 and may define the second path, which corresponds to the perimeter portion (or boundary layer) of the vacuum field as described herein. Vacuum channels 123 may be open channels through which the vacuum product may flow and are created by including material between first cone 121 and second cone 122 to define each channel. The material used to separate the channels of vacuum channels 123 may also be used to connect first cone 121 to second cone 122. Alternatively, first cone 121 and second cone 122 may be connected in other ways. For example, first cone 121 and second cone 122 may each be connected to disconnect 127. In that embodiment, disconnect 127 may include an inner hollow chamber, which is used to allow the inner portion of the vacuum product to flow into disconnect 127 from the first cone 121. First cone 121 is connected to disconnect outside of this hollow chamber. A second hollow chamber (i.e. a concentric annulus shape outside of the connection between disconnect 127 and first cone 121) may be formed in disconnect 127 to provide for flow from the second cone 122 (i.e. the flow between first cone 121 and second cone 122), and second cone 122 may be connected to disconnect outside of the second hollow chamber.

Vacuum channels 123 may follow a path P between first cone 121 and second cone 122 (i.e. a curved path, a straight path, etc.), which may define the second path and determine the flow of the outer, or perimeter, portion of vacuum field. While path P depicted in FIG. 1C is a single path shown for reference, each vacuum channel of vacuum channels 123 follows a path that is similar to path P (i.e. each vacuum channel has its own path, which collectively define the flow path of perimeter portion). In the embodiment depicted in FIG. 1C, path P is a curved path that may create a curved flow path within the perimeter portion of the vacuum field. Path P is depicted as a single vacuum channel for reference. In practice, there are multiple vacuum channels (when used), which generally follow the same, similar paths. This curved path P may help limit and/or prevent aerosols generated by the patient, which should reside within the inner portion of the vacuum field, from exiting the vacuum field, as the boundary layer tends to maintain these aerosols within the inner portion of the flow and into hood 120. The perimeter portion of the vacuum field may capture aerosols from the patient and may also, or alternatively, create a boundary layer limits and/or prevents aerosols from a patient from reaching a dentist, hygienist, etc. and vice versa. The curved path P may cause the boundary layer (or perimeter portion) to more effectively limit and/or prevent aerosols from a patient from reaching a dentist and vice versa. In this way, the outer portion of the vacuum field may limit and/or prevent the patient and dentist (hygienist, etc.) from being exposed to the aerosols of one another. While hood 120 depicts vacuum channels that 123 define the flow of the vacuum product through the outer portion, other embodiments may not include vacuum channels.

As shown in FIG. 1C, first cone 121 may terminate at a first end 125 of dental hood 120, and second cone 122 may extend past first end 125 to terminate at second end 126. Second cone 122 may be flared between first end 125 and second end 126, which may influence the perimeter portion of vacuum field. Additionally, or alternatively, the flare may begin at a different point (i.e. rather than first end 125). Light 124 (one light, two lights, LED light, incandescent lights, etc.) may be included on dental hood 120, which may permit dental hood to provide light to a patient (i.e. in connection with, or substitution of, the traditional dental light. Light 124 is depicted as a ring of LED (light emitting diode) lights around the perimeter of the end (i.e. the end opposite disconnect 127), but light 124 may be any number of configurations. A disconnect 127 (threaded end, quick disconnect, end that may be tightened to duct or valve with a clamp, etc.) may be located on dental hood 120, which may allow dental hood 120 to be easily removed from a collector (from a duct, a valve, etc.), such as for cleaning, changing, etc. Disconnect 127 may be connected to first cone 121 and/or second cone 122. In the embodiment depicted in FIG. 1C, disconnect 127 is connected to second cone 122, and vacuum channels 123 both define the path P and connect first cone 121 and second cone 122. Disconnect 127 is a hollow member that allows the vacuum product to be pulled in through hood 120 and into the ducts, valves, etc. upsteam of hood 120. Handle 112 is depicted in FIG. 1C as connected to disconnect 127, but may be connected to another portion of hood 120 and/or another portion of a collector (i.e. a valve, duct, etc.).

In practice, hood 120 receives a negative, or vacuum, pressure that is generated by a source (a vacuum pump or fan, such as vacuum 230, etc.) which creates a vacuum field (i.e. a volume within which the flow of air and airborne particulates is directed into hood) from which the vacuum product flows into hood 120. The vacuum is introduced to hood 120 at disconnect 127. The vacuum product is pulled through first cone 121 and second cone 122 (i.e. the area between first cone 121 and second cone 122) and, as a result, each of first cone 121 and second cone 122 affect influence the vacuum field. When vacuum channels 123 are present, the flow between second cone 122 and first cone 121 follows the path of the vacuum channels 123, which may cause the perimeter portion, or boundary layer, of the flow path to be curved. Because the boundary layer includes a flow path that is different from the inner portion of flow (i.e. that portion of flow that passes through first cone 121), the aerosols captured within the inner portion may not escape the boundary layer, which prevents patient aerosols from reaching the dentist, hygienist, etc. Further, the boundary layer may “pull in” aerosols from the dentist, hygienist, etc., which limits and/or prevents such aerosols from being introduced to the patient.

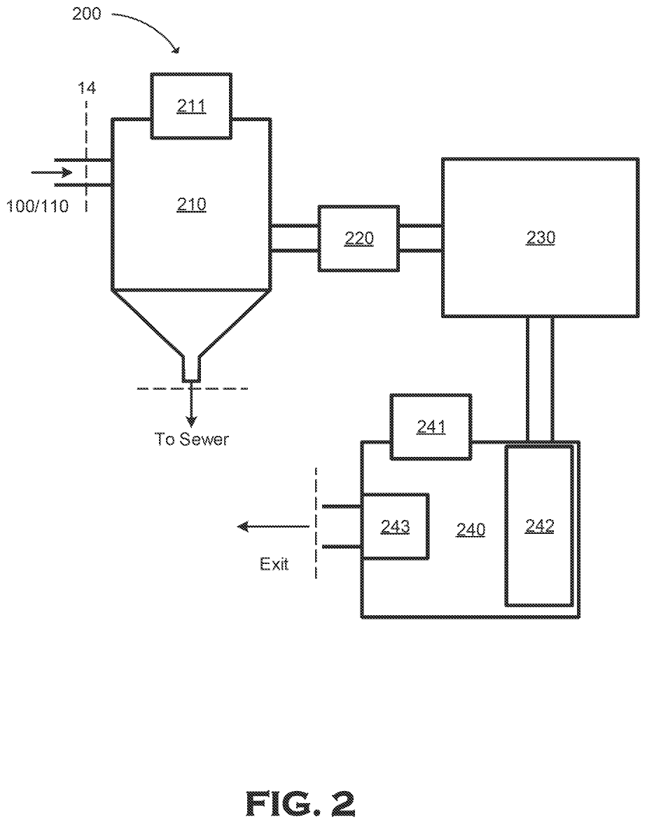

FIG. 2 illustrates an example embodiment of a capture system 200. As shown in FIG. 2, capture system 200 includes first collector 100, second collector 110, separator 200, filter 220, vacuum 230 and exhaust 240. In the arrangement depicted in FIG. 2, first collector 100 and second collector 110 may be on the opposite side of wall 14 from the remaining components of capture system 200. In other words, first collector 100 and second collector 110 are in separate rooms (the same room or different rooms) from the remaining components of capture system 200.

The remaining components of capture system may be centralized, meaning they are typically located together away from the procedure, patient and/or provider. In this arrangement, the processing of dental aerosols may take place away from the room in which a procedure takes place (for safety, noise reduction, etc.), but together, which may help with servicing, diagnostics, etc. The components described in relation to FIG. 2 are provided for explanatory purposes only, and the disclosure herein is not intended to be limited to the exact components, or arrangements of components, reflected in FIG. 2. There may additional components, fewer components and/or differently arranged components than what is shown in FIG. 2. Also, in other implementations, one or more of the components of FIG. 2 may perform the function of one or more other components of FIG. 2.

Separator 210 may separate liquid components of the vacuum product from the non-liquid products of the vacuum product. Separator 210 may be formed from any conventional water separator, including cyclone-type separators, p-traps, in-line separators, etc. Separator 210 may further include a decontaminator 211, which may reduce and/or eliminate any biohazards in vacuum product. Decontaminator 211 may include a UV-C light emitter, wet scrubber, or any other type of decontamination known in the art.

Filter 220 may filter the vacuum product after it passes through separator and before vacuum 230. Filter 220 may be any known filter product (HEPA filters, basic filters, ionizers, ozone generators, electrostatic filters, activated carbon filters, etc.). Vacuum 230 may be any type of vacuum unit that is known to generate a vacuum in a gas, such as a high capacity, low vacuum unit (e.g. a centrifugal blower, etc.) or a low capacity, high vacuum unit (e.g. positive displacement pumps, rotary claw, rotary screw, water pump, etc.).

Exhaust 240 may treat the vacuum product before it exits the capture system. Exhaust may include a chamber that includes a diffuser 242, which may diffuse the vacuum product. Exhaust 240 may also, or alternatively, include a second decontaminator 241, which may be the same as, similar to, or different from decontaminator 211. Decontaminator 241 may eliminate the biohazard in vacuum product before it exits exhaust 240. Exhaust 240 may include a powered exhaust fan 243, which may limit and/or prevent backpressure imposed on vacuum 230. Exhaust 240 may discharge the vacuum product somewhere safe, such as outside, in a closet, etc.

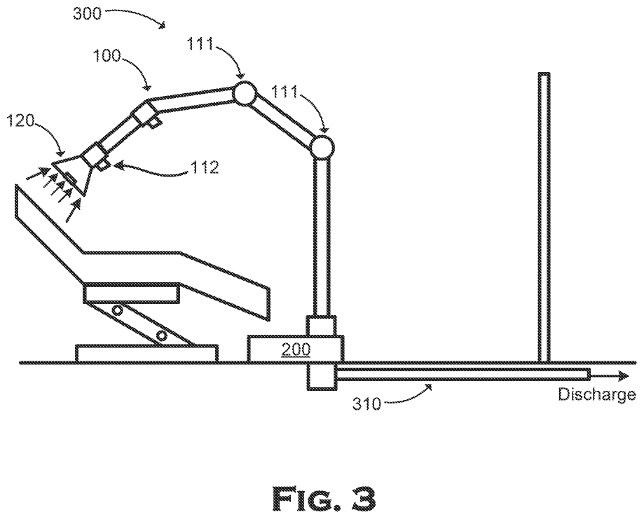

FIG. 3 depicts an alternative environment in which the systems and/or methods, described herein, may be implemented. As shown in FIG. 3, environment 300 may include the first collector 100 of FIG. 1, which is connected to the remaining components of capture system 200, such as by extending therefrom. A discharge line 310 carries the vacuum product from the capture system 200 and out of environment 300 after it has been treated.

The foregoing description provides illustration and description, but is not intended to be exhaustive or to limit the implementations to the precise form disclosed herein. Modifications and variations are possible in light of the above disclosure or may be acquired from practice of the disclosed embodiments. It will be apparent that the systems and/or methods described herein may be implemented in many different forms and in many different ways in addition to those specifically described with reference to FIGS. 1A through 3. For example, the capture systems described herein may be implemented in areas other than dental offices. The actual components and/or materials used to implement the embodiments reflected in FIGS. 1A through 3 are merely example materials and may be used in any alternative environment to provide shade as described herein. It should be understood that the components and/or materials may be designed in any form or fashion to meet the requirements of a particular embodiment.

It should be emphasized that the terms comprises/comprising when used in this specification are taken to specify the presence of stated features, integers, steps or components, but does not preclude the presence or addition of one or more other features, integers, steps, components or other groups thereof.

What is claimed is:

1. An extra-oral capture hood comprising:
a first cone that provides a first path for a vacuum product through the extra-oral capture hood,
a second cone concentric with the first cone and disposed around the first cone, the second cone providing a second path for the vacuum product through the extra-oral capture hood, the second path being located between the first cone and second cone;
a disconnect connected to at least one of the first cone or second cone, the disconnect configured to connect the extra-oral capture hood to a duct, a valve or other component of a capture system.

2. The extra-oral capture hood of claim 1, where the first cone includes a first end opposite the disconnect and the second cone includes a second end opposite the disconnect, the second end extending past the first end.

3. The extra-oral capture hood of claim 2, where the second cone is flared between the first end and the second end.

4. The extra-oral capture hood of claim 1 further comprising vacuum channels between the first cone and second cone, the vacuum channels further defining the second path.

5. The extra-oral capture hood of claim 4, where the vacuum channels have a curved path.

6. The extra-oral capture hood of claim 4, where a material used to form the vacuum channels connects the first cone to the second cone.

7. The extra-oral capture hood of claim 1 further including a light.

8. The extra-oral capture hood of claim 6 where the light includes a ring of LED lights located around the second cone.

9. The extra-oral capture hood of claim 1 further comprising a handle.

10. The extra-oral capture hood of claim 1 where the disconnect includes a quick disconnect.

11. An extra-oral capture system for capturing a vacuum product comprising:
a hood comprising a first cone, a second cone and a disconnect, the first cone defining a first path for the vacuum product and a second path for the vacuum product being defined between the first cone and second cone,
a valve connected to the disconnect, the valve configured to adjust the flow of the vacuum product;
a duct connected to valve; and
a vacuum configured to provide a vacuum pressure to the duct, the vacuum pressure causing the vacuum product to enter the hood.

12. The extra-oral capture system of claim 11 further including a second hood connected to the duct.

13. The extra-oral capture system of claim 11 where the hood is located in a first room and the vacuum is located in a second room.

14. The extra-oral capture system of claim 11, where the first cone includes a first end opposite the disconnect and the second cone includes a second end opposite the disconnect, the second end extending past the first end.

15. The extra-oral capture system of claim 11, where the second cone is flared between the first end and the second end.

16. The extra-oral capture system of claim 11, where the hood includes a light.

17. The extra-oral capture system of claim 11, further comprising vacuum channels between the first cone and second cone, the vacuum channels further defining the second path.

18. The extra-oral capture system of claim 17, where the vacuum channels are curved.

19. The extra-oral capture system of claim 17 where a material used to form the vacuum channels connects the first cone to the second cone.

20. The extra-oral capture system of claim 11 further including a reservoir including a decontamination fluid, the reservoir being configured to store the hood in the decontamination fluid.
